(19) United States
(12) Patent Application Publication
(10) Pub. No.: US 2020/0338019 A1
(43) Pub. Date: Oct. 29, 2020

(54) TOPICAL FORMULATIONS CONTAINING VITAMIN A AND USES THEREOF

(71) Applicant: Vinod Tawar, Langley (CA)

(72) Inventor: Vinod Tawar, Langley (CA)

(21) Appl. No.: 16/859,638

(22) Filed: Apr. 27, 2020

Related U.S. Application Data
(60) Provisional application No. 62/839,718, filed on Apr. 28, 2019.

(51) Int. Cl. A61K 31/07; A61K 31/355; A61K 47/44; A61K 47/24; A61K 31/65; A61P 27/02; A61P 17/06; A61P 17/10

(52) U.S. Cl. CPC A61K 31/07; A61K 31/355; A61K 47/44; A61P 17/10; A61K 31/65; A61P 27/02; A61P 17/06; A61K 47/24

(57) ABSTRACT

The present invention relates to topical skin care formulations comprising vitamin A, typically as acetate of palmitate, vitamin E, almond oil, rose hip oil, evening primrose oil, coconut oil, jojoba oil, gardenia fragrance oil, PLO gel, and tetracyclines or macrolides. The medicinal uses may also include, but are not limited to eczema, acne, psoriasis, atopic dermatitis, rosacea, and dermatitis, but may include the topical treatment of retinal disorders consisting of: glaucoma, corneal dysfunction, adult macular degeneration, cataracts, and diabetic retinopathy. The topical formulations may also be used to treat carcinogenic effects via anti-inflammatory and immunogenic properties. The cosmetic uses may include, but are not limited to, anti-wrinkle effects as an incident of aging.

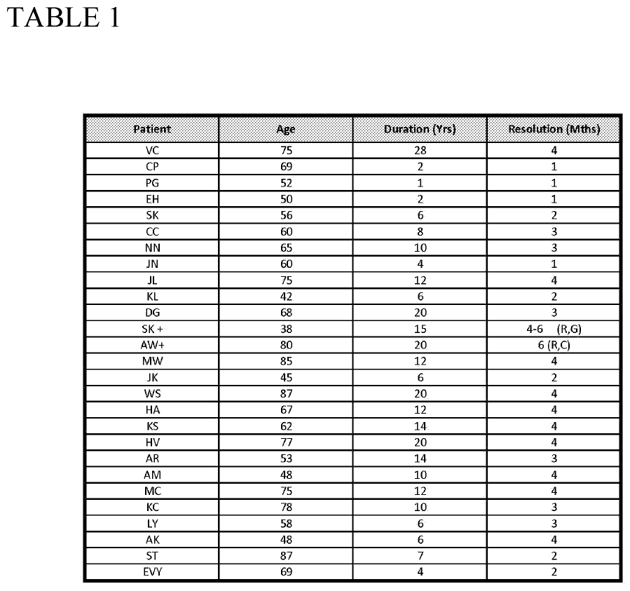

### CROSS-REFERENCE TO RELATED APPLICATIONS

This application claims priority to U.S. Provisional Application No. 62/839,718, filed Apr. 28, 2019, which is hereby incorporated by reference herein in its entirety.

### FIELD OF THE INVENTION

The present disclosure relates to topical formulations, particularly to formulations used in the field of skin care and more particularly to therapeutic uses of formulations comprising vitamin A, vitamin E, and various essential oils and mild fragrances and combinations thereof.

### BACKGROUND

Skin care has been practiced for thousands of years, dating back to the Ancient Egyptians. Cleopatra was known for her skin care regimen and is said to have discovered some of the first anti-aging methods. Skin care has evolved through the years with every generation being eager to slow, prevent or reverse the aging process, prevent acne, and heal skin. Countless skin care products are commercially available for beautification of the skin and to fight wrinkle formation.

The effectiveness of all skin care products is normally contingent upon delivery of the active ingredients therein through the stratum corneum and viable epidermis into the dermis layer of the skin structure. This is because the active ingredients in the skin care product cannot be effective unless they penetrate through the dead layers of skin tissue and into the dermis layer of living skin cells. This is normally a difficult proposition for water soluble active ingredients, such as ascorbic acid, because the stratum corneum is a good water barrier. The stratum corneum and viable epidermis act to protect the body by holding water therein to prevent dehydration and by keeping external water which is frequently contaminated out of the body.

Retinoids are a group of compounds that include retinol (Vitamin A), retinal, numerous retinoic acids, esters and similar derivatives. Many retinoids have useful skin-treatment properties and have been used extensively to treat acne vulgaris. However the use several compounds have been limited because of their irritancy or toxicity when administered in excess. Other compounds are unstable under exposure to heat, oxygen, and ultraviolet light of have highly teratogenic and mutagenic effects.

Treatment of acne and other skin conditions can be difficult. A suitable solution is desired. Various attempts have been made to solve problems found in topical formulations for skin care art. Among these are found in: U.S. Patent and Publication U.S. Pat. Nos. 8,912,175; 4,885,311; and 2002/0155180. This prior art is representative of topical formulations for skin care.

None of the above cited documents, taken either singly or in combination, is seen to describe embodiments of the invention as claimed. Thus, a need exists for a reliable topical formulation for skin care, and to avoid the above-mentioned problems.

### BRIEF SUMMARY

It is an object of the invention to provide topical formulations containing vitamin A and uses thereof.

In accordance with an aspect of the invention there is provided a topical formulation comprising: one or more of vitamin A, derivatives or analogs thereof, vitamin E, derivatives or analogs thereof; one or more oils selected from almond oil, rose hip oil, evening primrose oil, coconut oil, jojoba oil; and gardenia fragrance oil; and Pluronic Lecithin Organogel.

In accordance with another aspect of the invention there is provided a method for treating a skin condition selected from the group consisting of acne vulgaris, cystic acne, eczema, psoriasis, atopic dermatitis, rosacea, dermatitis, hyper-pigmentation, dermal hypoplasia, epidermal hypoplasia, dermal keratosis, epidermal keratoses, wrinkles of the skin as an incident of aging, enlarged pores, surface roughness, ichthyoses, follicular disorders, benign epithelial tumors, perforated dermatoses, disorders of keratinization, and combinations thereof, comprising topically applying to skin affected by the skin condition, a composition comprising: one or more of vitamin A, derivatives or analogs thereof, vitamin E, derivatives or analogs thereof; one or more oils selected from almond oil, rose hip oil, evening primrose oil, coconut oil, jojoba oil; and gardenia fragrance oil; and Pluronic Lecithin Organogel.

In accordance with an additional aspect of the invention there is provided a method for treating retinal disorders selected from the group consisting of, glaucoma, corneal dysfunction, adult macular degeneration, cataracts, diabetic retinopathy and combinations thereof, comprising topically applying to skin surrounding an ocular region, a composition comprising: one or more of vitamin A, derivatives or analogs thereof, vitamin E, derivatives or analogs thereof; one or more oils selected from almond oil, rose hip oil, evening primrose oil, coconut oil, jojoba oil; and gardenia fragrance oil; and Pluronic Lecithin Organogel.

The advantages and features of the present invention will become better understood with reference to the following more detailed description and claims.

### BRIEF DESCRIPTION OF THE DRAWINGS

The FIGURE which accompanies the written portion of this specification illustrate embodiments and method(s) of use for the present invention, skin treatment topical formulation, constructed and operative according to the teachings of the present invention. Table 1 is a summary of the patient demographics relating to a clinical trial using a composition of a skin treatment topical formulation according to an embodiment of the present invention.

### DETAILED DESCRIPTION

Formulations and methods for carrying out the invention are presented in terms of embodiments described herein. However, the invention is not limited to the described embodiments, and a person skilled in the art will appreciate that many other embodiments of the invention are possible without deviating from the basic concept of the invention, and that any such work around will also fall under scope of this invention. It is envisioned that other styles and configurations of the present invention can be easily incorporated into the teachings of the present invention, and the configurations shall be shown and described for purposes of clarity and disclosure and not by way of limitation of scope.

Embodiments of the present invention provide a safe and effective combination of active agents characterized by synergistic activity, and which is particularly useful in the treatment of skin disorders and the topical treatment of retinal disorders. The combined active agents include at least one retinoid, particularly selected from a class of retinoids that are non-irritating to skin, and a combination of vitamins and essential oils and mild fragrances. The combination of these active ingredients provides a skin treatment formulation characterized by rapid onset of activity and lack of skin irritation.

The present invention describes topical formulations and their cosmetic and medicinal uses. The formulations consist of various derivatives, analogs and isomers of vitamin A. Specifically retinyl acetate and retinyl palmitate. The formulations may contain various different active and non-active components including derivatives, analogs and isomers of vitamin E and various essential oils and mild fragrances. The non-active base of the formulation may be a cream, foam, gel, lotion or an ointment.

The medicinal uses may include, but are not limited to, the topical treatment of skin disorders consisting of: eczema, acne, psoriasis, atopic dermatitis, rosacea, and dermatitis.

The medicinal uses may also include, but are not limited to, the topical treatment of retinal disorders consisting of: glaucoma, corneal dysfunction, adult macular degeneration, cataracts, and diabetic retinopathy.

The medicinal uses may also include, but are not limited to, anticarcinogenic effects via anti-inflammatory and immunogenic properties.

The cosmetic uses may include, but are not limited to, anti-wrinkle effects as an incident of aging.

Embodiments of the present invention are directed to skin treatment topical formulations for dermatological conditions for external application to the affected areas 1-2×/day. The objective of topical formulations is to overcome systemic adverse effects. The formulations are effective in the treatment of challenging illnesses, requiring additional treatments for adverse effects from oral or injectable forms in addition to the extreme cost.

In one embodiment of the present invention, skin treatment topical formulations may comprise vitamin A acetate U.S.P, vitamin E, almond oil, rose hip oil, evening primrose oil, coconut oil, jojoba oil, gardenia fragrance oil, PLO gel, and tetracyclines or macrolides 1-1.5% w/w.

In an additional embodiment of the present invention, skin treatment topical formulations may comprise vitamin A palmitate U.S.P, vitamin E, almond oil, rose hip oil, evening primrose oil, coconut oil, jojoba oil, gardenia fragrance oil, PLO gel, and tetracyclines or macrolides 1-1.5% w/w.

The topical embodiments of the present invention are typically formulated in PLO gel also known as Pluronic Lecithin Organogel. PLO gel is a mixture of oil and water that thickens to form a gel comprising lecithin, and isopropyl palmitate.

Skin treatment topical formulation may be used for treatment of acne, eczema, psoriasis atopic dermatitis and other illnesses via topical formulations consisting of retinyl acetate with or without antibiotics. Retinyl acetate U.S.P. or retinal palmitate in adequate concentration as permitted (0.1% to 1.85% w/w) is mixed with vitamin E and various essential oils, mild fragrance supplemented with a base consisting of lipophilic/lipophobic products available from compounding suppliers. The application of antibiotics incorporation depends on results observed. In treatment resistant cases, antibiotics may be added. A concentration of antibiotic ranges from 1-4%. The duration of such treatment is limited to 8-12 weeks to avoid incidences of microbial resistance.

An alternative embodiment of the composition may include retinyl acetate or retinyl palmitate 0.3-1.85% w/w, and optionally an antibiotic 1-4% (tetracyclines or macrolides).

An alternative embodiment of the composition may include retinyl acetate or retinyl palmitate 1.50-1.85% w/w, and optionally an antibiotic 1-4% (tetracyclines or macrolides).

An alternative embodiment of the composition may include retinyl acetate or retinyl palmitate 1.5 w/w and optionally an antibiotic 1-4% (tetracyclines or macrolides).

Formulation 1A:

Retinyl acetate U.S.P (0.1-1.85% w/w), vitamin E (0.1-1.85% w/w), tetracyclines or macrolides (1.0-4.0% w/w), and one or more oils selected from: almond oil, rose hip oil, evening primrose oil, coconut oil, jojoba oil, gardenia fragrance oil, in a PLO gel.

Formulation 1B:

Retinyl acetate U.S.P (1.5% w/w), vitamin E (1.0% w/w), and optionally tetracyclines or macrolides (1.0% w/w), and one or more oils selected from: almond oil, rose hip oil, evening primrose oil, coconut oil, jojoba oil, gardenia fragrance oil, in a PLO gel

Formulation 2A:

Retinyl palmitate U.S.P (0.1-1.85% w/w) vitamin E (0.1-1.85 w/w), optionally including tetracyclines or macrolides (1.0-4.0% by weight), and one or more oils selected from: almond oil, rose hip oil, evening primrose oil, coconut oil, jojoba oil, gardenia fragrance oil, in a PLO gel.

Formulation 2B:

Retinyl palmitate U.S.P (1.5% w/w), vitamin E (1.0% w/w), optionally including tetracyclines or macrolides (1.0% w/w), and one or more oils selected from: almond oil, rose hip oil, evening primrose oil, coconut oil, jojoba oil, gardenia fragrance oil, in a PLO gel.

Treatment of Diabetic Retinopathy and Other Retinal Disorders.

Disorders associated with diabetes commonly include a decreased vision to blindness and cataract formation. A focus of this study was to determine a beneficial role of vitamin A applied through the principle of transdermal absorption. The cream based topical Formulation 2B was used in this study.

The objective was to avoid having systemic adverse effects. The duration of treatments was found to be optimal for 12-16 weeks. Twenty seven subjects, or both genders, were studied for a duration of 12-16 weeks and periodically visual acuity was assessed by using Snellen charts in the office or symptoms evaluations, in cases of patients with retinal injuries. The results obtained was patients showing increased visual acuity mainly in daytime vision (cone regeneration).

Preparation of Formulation 2B—Vitamin A in the form of retinyl palmitate was weighed to render a concentration as permitted by Health Canada. In addition, various essential oils were added and incorporated into a lip balm base at a warmer temperature to provide a homogeneous mixture. Samples weighing 12 grams. Each were given to patients on trial and they were seen in a follow-up every 4 weeks.

The mode of administration was applying Formulation 2B twice a day externally on upper and lower eye lids. Assessments of visual acuity changes were determined by Snellen chart, and in cases of retinal injuries ophthalmological consultation was done.

The parameters of study considered are age of the patient, duration of diabetes the patient has endured and resolution of symptoms (time required of treatment to see improvements in visual acuity to a near normal level). A duration of treatment needed for a satisfactory improvement may have been dependent on the level of glycemic control i.e. Sub-optimal control would reflect a longer need of treatment. In addition, rg represents patients with retinal injuries or glaucoma and rc includes a patient with retinitis and corneal injuries.

Formulation 2B application was found to be free of any side effects. In rare cases one may exhibit hypersensitivity by having erythema in peri-orbital region and or burning sensation. Patients were advised to discontinue using the product in those cases. However, hypersensitivity was of a rare occurrence. As described earlier the extent of treatment needed were variable. Most of the study participants demonstrated visual improvement to a near normal level in 3 to 4 months.

Significant indication of improvement in visual activity mainly daytime cone regeneration based on 4 months observations.

Table 1 indicates the demographics of the patients enrolled in the clinical study.

Cases with most remarkable improvement:

A 75 yrs. Old male with a history of retinal detachment 7 years earlier and resulting blindness, in spite of 4 surgeries, had seen a normal day time vision after a 4 month application of Formulation 2B.

A 57 years old female was diagnosed with retinal injury causing diminished vision and was scheduled for a surgery. She had a full recovery after 12 weeks of using the cream and was informed of not requiring the surgery at the end since the injury had healed.

A 70 years old male had a one-week history of diminishing vision from an acute retinal injury in one of his eyes and had experienced full relief of symptoms after using the cream for next 2 weeks.

Formulation 1A and Formulation 2A were initially formulated for the treatment of various forms of dermatitis (resistant to oral treatments), glaucoma, adult macular degeneration and cataracts prevention. The Formulations have also been the subject of clinical observations have shown beneficial role in eczema, acne, psoriasis, atopic dermatitis, rosacea, and dermatitis.

The skin treatment topical formulation includes a composition used for treatment of acne, eczema, psoriasis atopic dermatitis and other illnesses. The composition may be created using vitamin A acetate 1-1.5% (0.5 ml each), and vitamin E in equal proportion to vitamin A acetate. Antibiotics may be included in the composition for effective treatment. The application of antibiotics depends on case results observed. In treatment resistant cases, antibiotics may be added. A concentration of antibiotic may range from 1-4%. The duration of such treatment is limited to 8-12 weeks to avoid incidences of microbial resistance.

The foregoing descriptions of specific embodiments of the present invention have been presented for purposes of illustration and description. They are not intended to be exhaustive or to limit the invention and method of use to the precise forms disclosed. Many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient, but is intended to cover the application or implementation without departing from the spirit or scope of the claims of the present invention.

What is claimed is:

1. A topical formulation comprising:
(i) one or more of vitamin A, derivatives or analogs thereof, vitamin E, derivatives or analogs thereof;
(ii) one or more oils selected from almond oil, rose hip oil, evening primrose oil, coconut oil, jojoba oil; and gardenia fragrance oil; and
(iii) Pluronic Lecithin Organogel.

2. The topical formulation of claim 1, additionally comprising tetracyclines or macrolides.

3. The topical formulation of claim 1, wherein the vitamin A, derivatives or analogs thereof is retinyl acetate.

4. The topical formulation of claim 1, wherein the vitamin A, derivatives or analogs is retinyl palmitate.

5. The topical formulation of claim 1, comprising retinyl acetate and vitamin E.

6. The topical formulation of claim 1, comprising retinyl palmitate and vitamin E.

7. The topical formulation of claim 1, comprising 1.50-1.85% w/w retinyl acetate or 1.50-1.85% w/w retinyl palmitate.

8. A method for treating a skin condition selected from the group consisting of acne vulgaris, cystic acne, eczema, psoriasis, atopic dermatitis, rosacea, dermatitis, hyper-pigmentation, dermal hypoplasia, epidermal hypoplasia, dermal keratosis, epidermal keratoses, wrinkles of the skin as an incident of aging, enlarged pores, surface roughness, ichthyoses, follicular disorders, benign epithelial tumors, perforated dermatoses, disorders of keratinization, and combinations thereof, comprising topically applying to skin affected by the skin condition, a composition comprising:
(i) one or more of vitamin A, derivatives or analogs thereof, vitamin E, derivatives or analogs thereof;
(ii) one or more oils selected from almond oil, rose hip oil, evening primrose oil, coconut oil, jojoba oil; and gardenia fragrance oil; and
(iii) Pluronic Lecithin Organogel.

9. The method of claim 8, wherein the composition comprises: retinyl acetate and vitamin E.

10. The method of claim 8, wherein the composition comprises: retinyl palmitate and vitamin E.

11. The method of claim 8, wherein the composition additionally comprises tetracyclines or macrolides.

12. The method of claim 8, wherein the composition comprises 1.50-1.85% w/w retinyl acetate or 1.50-1.85% w/w retinyl palmitate.

13. A method for treating retinal disorders selected from the group consisting of, glaucoma, corneal dysfunction, adult macular degeneration, cataracts, diabetic retinopathy and combinations thereof, comprising topically applying to skin surrounding an ocular region, a composition comprising:
(i) one or more of vitamin A, derivatives or analogs thereof, vitamin E, derivatives or analogs thereof;
(ii) one or more oils selected from almond oil, rose hip oil, evening primrose oil, coconut oil, jojoba oil; and gardenia fragrance oil; and
(iii) Pluronic Lecithin Organogel.

14. The method of claim 13, wherein the composition comprises: retinyl acetate and vitamin E.

15. The method of claim 13, wherein the formulation comprises: retinyl palmitate and vitamin E.

16. The method of claim 13, wherein the formulation additionally comprises tetracyclines or macrolides.

15. The method of claim 13, wherein the composition comprises 1.50-1.85% w/w retinyl acetate or 1.50-1.85% w/w retinyl palmitate.
